(19) United States
(12) Patent Application Publication
(10) Pub. No.: US 2019/0350778 A1
(43) Pub. Date: Nov. 21, 2019

(54) Diaper Tab Assembly With Z-Fold And Multiple Fastening Components

(71) Applicant: 3M INNOVATIVE PROPERTIES COMPANY, St. Paul, MN (US)

(72) Inventors: Shengguang Qi, Feicuilvzhou (CN); Thomas J. Gilbert, St. Paul, MN (US)

(21) Appl. No.: 16/476,996

(22) PCT Filed: Jan. 20, 2017
(86) PCT No.: PCT/CN2017/071892

§ 371 Date: Jul. 10, 2019

(51) Int. Cl. A61F 13/58; A61F 13/551

(52) U.S. Cl. CPC A61F 13/581; A61F 13/5633; A61F 13/55115

(57) ABSTRACT

An absorbent article (10) having an inner surface, an outer surface, a first waist region (32), a second waist region (34), a crotch region extending longitudinally between and connecting the first waist region (32) and the second waist region (34). Fastener tab (3) for an absorbent article (10) includes two hook-type strips of material, separated by an expansion area (39). The expansion area (39) has two folds (6, 7), creating a “Z-fold” type configuration. A strip of non-woven material which, when S-cut, may be used for the fastening tab region of an absorbent article (10).

### BACKGROUND

Diaper closure systems typically comprise tab assemblies fastened to one end of a diaper assembly construction at each lateral side of the diaper. The tabs may include a fastening component such as an area of pressure sensitive adhesive, or a system of hooks, which are designed to interface with, and couple to, a second fastening component included on the diaper body, such as a plastic surface or an area having a loop-type material (e.g., a knit-type fabric or nonwoven landing pad). Together, the first and second fastening components comprise a fastening system that is used to secure the diaper to a user's body.

Diaper tabs usually couple to the body of a diaper assembly in an area called the landing zone. An individual, sometimes a parent or care giver, pulls the diaper tab over and across the wearer's body and secures the diaper tab to the diaper body by pressing the tab assembly's first fastening component into the landing zone area (the tab assembly, so coupled to the landing zone, referred to herein as the user joint).

Such diaper fastening systems are releasable to allow both permanent removal of the diaper from the user's body, and to allow unfastening to inspect the diaper followed by refastening as necessary.

The fastener tape system may rely solely upon pressure-sensitive adhesive in the formation of the user joint as shown in U.S. Pat. Nos. 4,795,456, 4,710,190, 4,020,842 and 3,833,456. The use of combined adhesive and mechanical fastener systems is shown in U.S. Pat. Nos. 5,019,065, 5,053,028 and 4,869,724.

The use of extensible or stretchable tab assemblies to promote user comfort through better fit and more secure mounting is known in the art. The tabs operate as extensible diaper side waistbands. Examples of such diaper fastening systems are disclosed in U.S. Pat. Nos. 4,795,456, 4,066,081, 4,051,853 and 3,800,796. Related art includes U.S. Pat. Nos. 4,465,717, 4,662,875, 5,051,259, 5,106,384, 5,133,707, 5,531,731, 5,591,521, and 6,524,294.

### SUMMARY

A diaper tab assembly with a first side having two fastening components separated by an expansion area that includes two folds. The two folds comprise a Z-fold configuration. At the time of use, a user pulls the diaper tab assembly away from the diaper chassis to unfold it, thereby increasing its lateral dimension. The user then pulls the diaper tab assembly over and across a wearer, pressing the two hook-type fastening components into a non-woven loop-type material on the diaper's body, thereby securing it with a snug fit to the wearer's body.

The diaper tab assembly includes a tab chassis comprised of a suitable non-woven material, which may be comprised of one or more layers. The tab assembly is bonded to the diaper chassis using ultrasonic or adhesive coupling techniques known in the art.

The two fastening components on the diaper tab assembly may comprise a pressure sensitive adhesive, but preferably they comprise a hook-type material. A suitable material chosen to appropriately couple with the selected fastening component is on the outer front surface of the diaper body.

In selected embodiments, the second side of the diaper tab assembly (the side that does not include the two fastening components) may have a surface designed to couple with the first or second fastening component, such that the diaper tab assembly of the right or left side of the diaper may be installed on a wearer such that one overlaps the other.

The expansion area may, in some embodiments, comprise an elastomeric material.

In this application:

Terms such as “a”, “an” and “the” are not intended to refer to only a singular entity, but include the general class of which a specific example may be used for illustration. The terms “a”, “an”, and “the” are used interchangeably with the term “at least one”.

The phrase “comprises at least one of” followed by a list refers to comprising any one of the items in the list and any combination of two or more items in the list. The phrase “at least one of” followed by a list refers to any one of the items in the list or any combination of two or more items in the list.

The term “machine direction” (MD) as used herein denotes the direction of a running, continuous web during the manufacturing of the absorbent article disclosed herein. In a roll, for example, comprising a carrier web and a fastening strip, the machine direction corresponds to the longitudinal direction of the roll. Accordingly, the terms machine direction and longitudinal direction may be used herein interchangeably. The term “cross-direction” (CD) as used herein denotes the direction that is essentially perpendicular to the machine direction. When a portion of the laminate disclosed herein is cut from a roll, the cross-direction corresponds to the width of the roll.

The terms “first”, “second”, and “third” are used in this disclosure. It will be understood that, unless otherwise noted, those terms are used in their relative sense only. For these components, the designation of “first”, “second”, and “third” may be applied to the components merely as a matter of convenience in the description of one or more of the embodiments.

All numerical ranges are inclusive of their endpoints and nonintegral values between the endpoints unless otherwise stated.

### BRIEF DESCRIPTION OF DRAWINGS\

FIG. 1 is a drawing of a diaper having fastening tabs.

FIG. 2 is a drawing of a diaper with fastening tabs closing the diaper assembly (as fastened around the waist of a user).

FIG. 3 is a drawing of fastening components of a fastening tab and the adjacent landing zone of the diaper chassis.

FIG. 4 is a plan view of a fastening tab.

FIG. 5 is a profile view of the fastening tab of FIG. 4, in a partially extended state.

FIG. 6 is a profile view of the fastening tab of FIG. 4, in a folded state.

FIG. 7a is a plan view of a strip of manufactured tab material before it is Z-folded and placed on a roll.

FIG. 7b is a profile view of a strip of manufactured tab material of FIG. 7a.

FIG. 8a is a plan view of the strip of manufactured tab material of FIG. 7, in a Z-folded state.

FIG. 8b is a profile view of one of a fastening tabs resulting from conversion of the manufactured tab material of FIG. 8a.

FIG. 8c is a profile view of one of the fastening tabs resulting from conversion of the manufactured tab material of FIG. 8a.

FIG. 9 is a drawing of a diaper chassis having Z-folded fastening tabs coupled thereto (in folded state).

FIG. 10a is a plan view of one of the fastening tabs of FIG. 9 in an extended (unfolded) state.

FIG. 10b is a plan view of one of the fastening tabs of FIG. 9 in an extended (unfolded) state.

In the figures, like reference numerals designate like elements.

### DETAILED DESCRIPTION

The present disclosure relates generally to absorbent articles intended for personal wear, and more particularly to disposable absorbent articles having elongated, Z-folded tab assemblies that include two fastening components, allowing a user to selectively fasten and refasten the article about the wearer. In some embodiments such a design of the tab assemblies allows for a more secure, snug fit, which may be particularly useful in challenging closure environments, as may be encountered with premature or smaller babies.

Many absorbent articles intended for personal wear, such as diapers, training pants, feminine hygiene products, adult incontinence products, bandages, medical garments and the like are designed to be sufficiently absorbent to absorb moisture from liquid body exudates including urine, menses, blood, etc., away from the wearer to reduce skin irritation caused by prolonged wetness exposure. Diapers, as an example, are typically placed and secured on a wearer using a set of primary fastening tabs, such as adhesive tabs or mechanical (e.g., hook or loop) fastening system tabs, and left in place to absorb insults as well as to contain fecal waste.

For articles where the attachment is refastenable, such as diapers and some training pants, so called pop-open events (separation of the fasteners) can sometimes occur as a result of stresses placed on the attachment by movement of the wearer. For example, and particularly for absorbent articles employing only one fastening system, as an infant or other wearer of the absorbent article moves about (e.g., crawls, walks, runs, bends, etc.) the shear stress placed on the fastening system due to the infant's movement may cause fastening tabs or the like to loosen or even come unfastened completely, resulting in an absorbent article which tends to leak, sag, or fall off of a wearer.

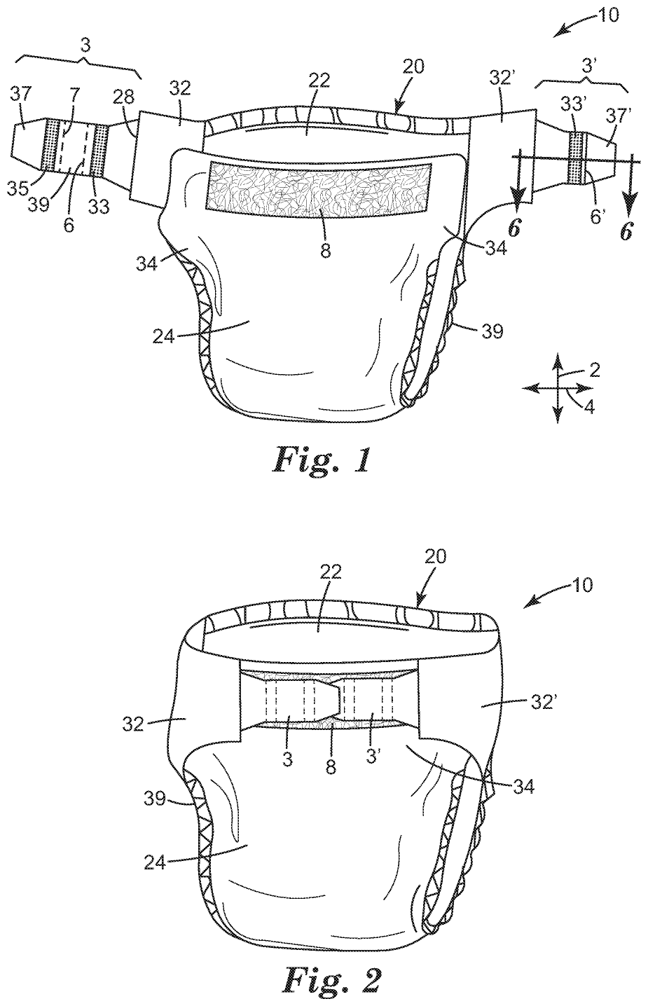

Accordingly, some known absorbent articles comprise more than one fastening system and/or fasteners to reduce the likelihood pop-open events, or incidences of the article leaking, sagging, falling off the user, etc. For example, US Patent Application Publication No. US2014/0142533 shows in its FIG. 1 an absorbent article having two longitudinally separated hook-type fastening systems on each of the two tabs. However, even with two such fastening system, current designs still may not adequately prevent pop-open type events, and may not provide the flexibility and conformability demanded by the market.

Against this background, it has now been discovered a novel design for a fastening tab assembly that is Z-folded and contains two (or more) fastening components separated by an expansion area. The Z-fold aspect of the design allows for a greater lateral extension of the tab assembly, allowing a care giver or in some cases the wearer him or herself to lift the tab assembly up and away from the absorbent article (thereby unfolding it), and pull it over and across the diaper's front lateral section and onto a receptive area of the diaper body, effecting a secure, comfortable user joint.

The two fastening components, separated by an expansion area, allow for improved conformability and sheer resistance in some embodiments. For example, in one embodiment further described below, the side of the fastening tab opposite the side having the fastening components may itself be configured for coupling to the fastening component—for example in the case of a hook-type fastening component, the opposite side of the tab assembly may comprise a loop-type nonwoven. In such an embodiment, the tab assembly may be pulled laterally to overlap and bond to the outer surface of the opposing tab assembly, a feature that may be particularly useful with small babies.

Absorbent articles according to the present disclosure include diapers and adult incontinence articles, for example. A schematic, perspective view of one embodiment of an absorbent article 10 according to the present disclosure and/or made according to a method of the present disclosure is shown in FIG. 1. Absorbent article 10 includes a chassis 20 with a topsheet side 22 (the bodyside liner defining the inner surface of the article as worn by a user) and a backsheet side 24 (the outer cover defining the outer surface of the article as worn by a user). The chassis 20 also has first and second opposing longitudinal edges 26 and 28 extending from a rear waist region 32 to an opposing front waist region 34. Front waist region 34 includes landing zone 8, which may comprise a material different from that of front waist region 34. In some embodiments landing zone 8 is designed to interface with fastening components that are part of tab assemblies that extend from the rear waist region 32. For example, if a hook-type material is used as a fastening component on tab assemblies 7 and 7′, landing zone 8 would comprise a loop-type material that acts as the associated fastening component (together the two fastening components comprising a fastening system), which effectively couples to the hook-type material when pressed by the user. An absorbent core is sandwiched between the topsheet side and backsheet side (not shown in FIG. 1).

The longitudinal direction of the absorbent article 10 refers to the direction extending between the rear waist region 32 and the front waist region 34, in the direction associated with longitudinal arrow element 2. Therefore, the term “longitudinal” refers to the length of the absorbent article 10, for example, when it is in an open configuration. The transverse direction of the absorbent article 10 refers to the direction extending between the two opposing edges of tab assemblies 3 and 3′, in the direction associated with transverse arrow element 4. Therefore, the term “transversely” refers to the width of the absorbent article 10, for example, when it is in an open configuration.

At least one of the front waist region 34 or the rear waist region 32, more typically the rear waist region 32, comprises tab assemblies (3 and 3′). Tab assembly 3 is shown in an unfolded configuration, and tab assembly 3′ is shown in a Z-folded configuration (discussed further below).

Tab assemblies 3 and 3′ comprise a tab chassis having one or more layers of non-woven material, bonded to transversely opposing ends of rear waist region 32 using techniques known in the art (e.g. adhesive or ultrasonic bonding). The tab assemblies may or may not be particularly elastomeric, in some cases relying on elastomeric properties of rear waist region 32 to achieve snugness against a user's body. Tab assembly 3, when unfolded as shown in FIG. 1, includes proximal fastening component 33 and distal fastening component 35, separated by an expansion area 39. Optional lifting tab 37 extends beyond distal fastening system 35. Expansion are 39 includes a proximal fold 6 and a distal fold 7 (in unfolded state, these folds are creases). As will be discussed further below, proximal and distal folds 6 and 7 combine to effect a Z-folded tab, shown as tab assembly 3′, which may be lifted and extended by a user griping and pulling lifting tab 37, thus unfolding distal folds 6 and 7 and in the process extending tab assembly from folded to unfolded state.

Tab assembly 3′ shows only proximal fold 6′, and a portion of distal fastening system 35′ (a portion of distal fastening system 35′ in this embodiment is obscured by the tab chassis associated with tab assembly 3′ in its folded state).

Absorbent articles (e.g., incontinence articles and diapers) according to the present disclosure may have any desired shape such as a rectangular shape, a shape like the letter I, a shape like the letter T, or an hourglass shape. The absorbent article may also be a refastenable pants-style diaper with laminates along each longitudinal edge. In some embodiments, the topsheet and backsheet are attached to each other and together form chassis 20 all the way out to the first and second longitudinal opposing edges 26 and 28. That is, the topsheet and backsheet together form the ear areas extending transversely and comprising the attachment area for tab assemblies 3 and 3′. In some embodiments, only one of the topsheet or the backsheet extends to the first and second longitudinal opposing edges 26 and 28. In other embodiments, the chassis can include separate side panels that are attached to the sandwich of at least topsheet, backsheet, and absorbent core during manufacturing of the absorbent article, for example, to form ear portions. The side panels can be made of a material that is the same as the topsheet or backsheet or may be made from a different material (e.g., a different nonwoven). In these embodiments, the side panels also form part of the chassis. In any of these embodiments, the absorbent article may comprise an elastic material 39 along at least a portion of first and second longitudinal side edges 26 and 28 to provide leg cuffs.

In absorbent articles according to the present disclosure and/or made according to the method of the present disclosure, the topsheet is typically permeable to liquid and designed to contact a wearer's skin, and the outwardly facing backsheet is typically impermeable to liquids. There is typically an absorbent core encased between the topsheet and the backsheet. Various materials can be useful for the topsheet, the backsheet, and the absorbent core in an absorbent article according to the present disclosure. Examples of materials useful for topsheets include apertured plastic films, woven fabrics, nonwoven webs, porous foams, and reticulated foams. In some embodiments, the topsheet is a nonwoven material. Examples of suitable nonwoven materials include spunbond or meltblown webs of fiber forming polymer filaments (e.g., polyolefin, polyester, or polyamide filaments) and bonded carded webs of natural polymers (e.g., rayon or cotton fibers) and/or synthetic polymers (e.g., polypropylene or polyester fibers). The nonwoven web can be surface treated with a surfactant or otherwise processed to impart the desired level of wettability and hydrophilicity. The backsheet is sometimes referred to as the outer cover and is the farthest layer from the user. The backsheet functions to prevent body exudates contained in absorbent core from wetting or soiling the wearer's clothing, bedding, or other materials contacting the diaper. The backsheet can be a thermoplastic film (e.g., a poly(ethylene) film). The thermoplastic film may be embossed and/or matte finished to provide a more aesthetically pleasing appearance. The backsheet can also include woven or nonwoven fibrous webs, for example, laminated to the thermoplastic films or constructed or treated to impart a desired level of liquid impermeability even in the absence of a thermoplastic film. Suitable backsheets also include vapor or gas permeable microporous “breathable” materials that are substantially impermeable to liquid. Suitable absorbent cores include natural, synthetic, or modified natural polymers that can absorb and hold liquids (e.g., aqueous liquids). Such polymers can be crosslinked (e.g., by physical entanglement, crystalline domains, covalent bonds, ionic complexes and associations, hydrophilic associations such as hydrogen bonding, and hydrophobic associations or Van der Waals forces) to render them water insoluble but swellable. Such absorbent materials are usually designed to quickly absorb liquids and hold them, usually without release. Examples of suitable absorbent materials useful in absorbent articles disclosed herein include wood pulp or other cellulosic materials and super absorbent polymers (SAP).

When the absorbent article shown 10 in FIG. 1 is worn by a user (e.g. a baby), the rear waist region 32 including tab assemblies 3 and 3′ may be extended from a Z-fold configuration, and wrapped around the wearer's body to overlap and engage with the front waist region 34 in the landing zone area 8 as shown in FIG. 2. In this configuration, the fastening components of tab assemblies 3 and 3′ face the backsheet and so would not be visible as shown in FIG. 2, but reference lines showing where fastening systems exist on the underside of such tab assemblies are shown for illustrative purposes. In some embodiments, the fastening components included in tab assemblies 3 and 3′ can engage with a receptive fastening component target area such as landing zone 8 comprising a fibrous material arranged on the backsheet of the front waist region 34. For example, loop tapes such as those disclosed in U.S. Pat. No. 5,389,416 (Mody et al.) EP 0,341,993 (Gorman et al.) and EP 0,539,504 (Becker et al.) may be applied to a target area to provide an exposed fibrous material. In other embodiments, the backsheet comprises a woven or nonwoven fibrous layer which is capable of interacting with the fastening component included on tab assemblies 3 and 3′. Examples of such backsheets 24 are disclosed, for example, in U.S. Pat. No. 6,190,758 (Stopper) and U.S. Pat. No. 6,075,179 (McCormack et al.). In these embodiments, the fastening component included in tab assemblies 3 and 3′ advantageously may engage with any suitable location on the backsheet (that is, a dedicated landing zone 8 is not necessary), which can be determined by the size of the wearer and the desired fit.

In the configuration shown in FIG. 2, tab assembly 3 overlaps tab assembly 3′. Though the overlap shown is not extensive enough to put fastening component of tab assembly 3 in contact with the backside of tab assembly 3′, such an embodiment is contemplated within the scope of this disclosure. In such an optional configuration, it is the first side of the tab assembly that has a surface that includes the fastening components; the side opposite the first die (the second side), then, is comprised of a surface selected to suitably engage with and couple to the tab assembly's fastening components. For example, the second side of tab assembly 3 may include a loop-type material designed to mechanically couple with a hook-type fastening component. Such an embodiment may be useful in circumstances where greater control of the articles diameter is desired, for example with small or premature babies. In other embodiments, the lateral dimension of the tab assembly is selected to not extend past the halfway point in the landing zone.

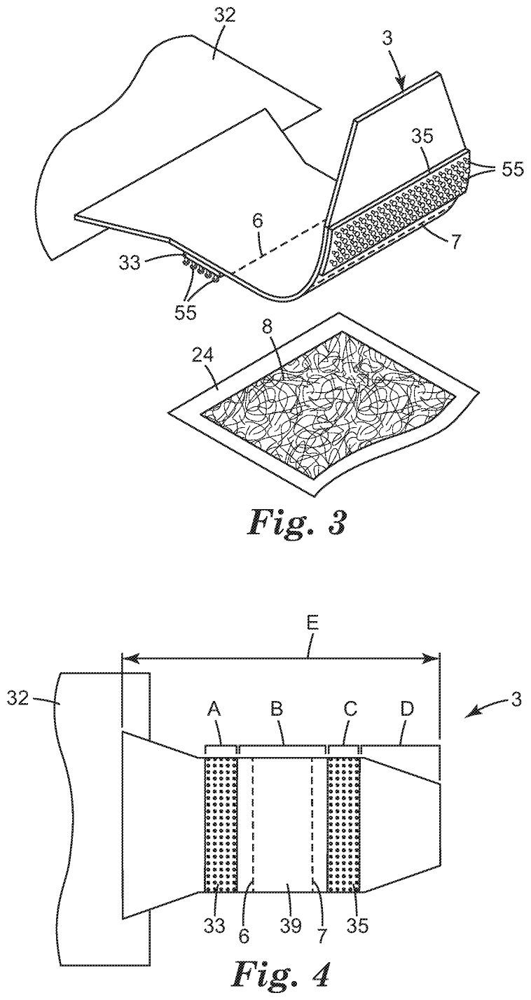

Turning now to FIG. 3, a three-dimensional rendering of tab assembly 3 is shown, along with a corresponding portion of landing zone 8, which will mechanically couple with proximal and distal fastening components included in tab assembly 3 upon a user pressing the two surfaces into one another. Rear waist region 32 is coupled to the tab chassis 50. As mentioned earlier, such coupling may be accomplished by means known in the art, such as via user of an adhesive system or by ultrasonic bonding techniques.

Tab chassis 50 is shown as a multi-layer construction, though single layer constructions or constructions having more than 2 layers are possible. Either layer may be continuous (i.e., without any through-penetrating holes) or discontinuous (e.g. comprising through-penetrating perforations or pores). Either layer of the tab chassis may comprise a variety of suitable materials including woven webs, non-woven webs (e.g., spunbond webs, spunlaced webs, airlaid webs, meltblown web, and bonded carded webs), textiles, paper, plastic films (e.g., single- or multilayered films, coextruded films, laterally laminated films, or films comprising foam layers), and combinations thereof. Any of these materials can be selected to be flexible enough to allow the tab chassis to be folded in a Z-configuration as discussed further herein.

In some embodiments, either or both layers in the tab chassis is a fibrous material (e.g., a woven, nonwoven, or knit material). In some embodiments, either layer comprises a nonwoven. The term “nonwoven” when referring to a tab chassis or web means having a structure of individual fibers or threads which are interlaid, but not in an identifiable manner as in a knitted fabric. Nonwoven fabrics or webs can be formed from various processes such as meltblowing processes, spunbonding processes, spunlacing processes, and bonded carded web processes. In some embodiments, the tab chassis comprises multiple layers of nonwoven materials with, for example, at least one layer of a meltblown nonwoven and at least one layer of a spunbonded nonwoven, or any other suitable combination of nonwoven materials. For example, either layer or both layers of the tab chassis may comprise a spunbond-meltbond-spunbond, spunbond-spunbond, or spunbond-spunbond-spunbond multilayer material. Or, the layers in the tab chassis may be a composite web comprising a nonwoven layer and a dense film layer (e.g., a thermoplastic film layer).

Fibrous materials that can provide useful layers of the tab chassis may be made of natural fibers (e.g., wood or cotton fibers), synthetic fibers (e.g., thermoplastic fibers), or a combination of natural and synthetic fibers. Exemplary materials for forming thermoplastic fibers include polyolefins (e.g., polyethylene, polypropylene, polybutylene, ethylene copolymers, propylene copolymers, butylene copolymers, and copolymers and blends of these polymers), polyesters, and polyamides. The fibers may also be multi-component fibers, for example, having a core of one thermoplastic material and a sheath of another thermoplastic material. In some embodiments, one or more zones of the tab chassis may comprise one or more elastically extensible materials extending in at least one direction when a force is applied and returning to approximately their original dimension after the force is removed. However, in some embodiments, at least the portion of the tab chassis joined to the fastening patch is not stretchable or has up to a 10 (in some embodiments, up to 9, 8, 7, 6, or 5) percent elongation in the CD. In some embodiments, the layers that comprise the tab chassis may be extensible but nonelastic. In other words, the tab chassis may have an elongation of at least 5, 10, 15, 20, 25, 30, 40, or 50 percent but substantially no recovery from the elongation (e.g., up to 10 or 5 percent recovery). Suitable extensible tab chassis material may include nonwovens (e.g., spunbond, spunbond meltblown spunbond, or carded nonwovens). In some embodiments, the nonwoven may be a high elongation carded nonwoven (e.g., HEC).

Useful layers used in the tab chassis may have any suitable basis weight or thickness that is desired for a particular application. For a fibrous tab chassis, the basis weight may range, e.g., from at least about 5, 8, 10, 20, 30, or 40 grams per square meter, up to about 400, 200, 100, or 50 grams per square meter. The tab chassis may be up to about 5 mm, about 2 mm, or about 1 mm in thickness and/or at least about 0.1, about 0.2, or about 0.5 mm in thickness.

Dashed lines are shown indicating the locations of proximal fold 6 and distal fold 7, both running longitudinally in the expansion area that comprises the tab chassis area between the proximal and distal fastening components (elements 33 and 35 respectively). In the embodiment shown in FIG. 3, both fastening components comprise patches of a hook-type material oriented longitudinally on the tab chassis 50. Other configurations are possible. Either proximal or distal fastening components could be a single discrete strip of material, or it could be an expanded strip of hook type material (as for example that shown with respect to FIG. 4 in 71022, hereby incorporated by reference in its entirety) or a plurality of narrow strips of spaced apart material which are grouped together to form a discreet fastening component. Regardless of the particular selection of fastening component, a first area of the tab assembly closest to the person (when extended laterally from the rear waist region) comprises the proximal fastening component 33, and the one further away, separated by an expansion area having two folds (proximal and distal folds 6 and 7 respectively) included therein, is the distal fastening component 35. Note that proximal and distal folds 6 and 7, when tab assembly is in extended state, are embodied as proximal and distal creases.

The hook type fastening components (elements 33 and 35) typically comprises upstanding male fastening elements 55 (and in break-out view, 55′) on a backing. The male elements are referred to herein as “hook type” but this terminology is used only in a general sense and is not intended to limit the design of the male elements; male elements may not actually include an actual hook-shape (for example they may be mushroom shaped or have other shapes). Hook type fastening component 57 is a profile view of a typical hook-type fastening component 33 and 35. It includes thermoplastic backing 56 and a plurality of male fastening elements 55. The male elements generally have a form that may be suitably loop engaging with a loop type material present in landing zone area 8 (referring back to FIG. 1). The backing and the male fastening elements 55 are typically integral (that is, formed at the same time as a unit, unitary). Hook-type fastening patches are typically made from at least one thermoplastic material. Suitable thermoplastic materials for mechanical fasteners include polyolefin homopolymers such as polyethylene and polypropylene, copolymers of ethylene, propylene and/or butylene; copolymers containing ethylene such as ethylene vinyl acetate and ethylene acrylic acid; polyesters such as poly(ethylene terephthalate), polyethylene butyrate and polyethylene napthalate; polyamides such as poly(hexamethylene adipamide); polyurethanes; polycarbonates; poly(vinyl alcohol); ketones such as polyetheretherketone; polyphenylene sulfide; and mixtures thereof. Typically, the thermoplastic is a polyolefin (e.g., polyethylene, polypropylene, polybutylene, ethylene copolymers, propylene copolymers, butylene copolymers, and copolymers and blends of these materials).

Upstanding male fastening elements on a backing can be made, for example, by feeding a thermoplastic material onto a continuously moving mold surface with cavities having the inverse shape of the posts. The thermoplastic material can be passed between a nip formed by two rolls or a nip between a die face and roll surface, with at least one of the rolls having the cavities. The cavities may be in the inverse shape of a capped post having a loop-engaging head or may be in the inverse shape of a post without loop-engaging heads (e.g., a precursor to a male fastening element). Pressure provided by the nip forces the resin into the cavities. In some embodiments, a vacuum can be used to evacuate the cavities for easier filling of the cavities. The nip typically has a large enough gap such that a coherent backing is formed over the cavities. The mold surface and cavities can optionally be air or water cooled before stripping the integrally formed backing and upstanding hook elements from the mold surface such as by a stripper roll. If the posts formed upon exiting the cavities do not have loop-engaging heads, loop-engaging heads could be subsequently formed into hooks by a capping method as described in U.S. Pat. No. 5,077,870 (Melbye et al.). Typically, the capping method includes deforming the tip portions of the hook elements using heat and/or pressure. The heat and pressure, if both are used, could be applied sequentially or simultaneously.

Suitable tool rolls include those formed from a series of plates defining a plurality of post-forming cavities about its periphery such as those described, for example, in U.S. Pat. No. 4,775,310 (Fischer). Cavities may be formed in the plates by drilling or photoresist technology, for example. Other suitable tool rolls may include wire-wrapped rolls, which are disclosed along with their method of manufacturing, for example, in U.S. Pat. No. 6,190,594 (Gorman et al.). Another exemplary method for forming a thermoplastic backing with upstanding posts includes using a flexible mold belt defining an array of upstanding post-shaped cavities as described in U.S. Pat. No. 7,214,334 (Jens et al.). Yet other useful methods for forming a thermoplastic backing with upstanding posts can be found in U.S. Pat. No. 6,287,665 (Hammer), U.S. Pat. No. 7,198,743 (Tuma), and U.S. Pat. No. 6,627,133 (Tuma).

Another method for forming a thermoplastic backing with upstanding male fastening elements is profile extrusion, which is described, for example, in U.S. Pat. No. 4,894,060 (Nestegard). Typically, in this method a thermoplastic flow stream is passed through a patterned die lip (e.g., cut by electron discharge machining) to form a web having downweb ridges. The ridges can then be transversely sliced at spaced locations along the extension of the ridges to form upstanding fastening elements with a small separation caused by the cutting blade. The separation between upstanding fastening elements is then increased by stretching.

The male fastening elements on the fastening patch of the laminate typically have loop-engaging heads that have an overhang. The term “loop-engaging” as used herein relates to the ability of a male fastening element to be mechanically attached to a loop material. Suitable male fastening elements with loop-engaging heads can have any desired shape. For example, the male fastening element may be in the shape of a mushroom (e.g., with a circular or oval head enlarged with respect to the stem), a hook, a palm-tree, a nail, a T, or a J. The loop-engageability of male fastening elements may be determined and defined by using standard woven, nonwoven, or knit materials. A region of male fastening elements with loop-engaging heads generally will provide, in combination with a loop material, at least one of a higher peel strength, higher dynamic shear strength, or higher dynamic friction than a region of posts without loop-engaging heads. Typically, male fastening elements that have loop-engaging heads have a maximum thickness dimension (in either dimension normal to the height) of up to about 1 (in some embodiments, 0.9, 0.8, 0.7, 0.6, 0.5, or 0.45) millimeter.

The male fastening elements on the fastening patch that comprises either the proximal or distal fastening component can have a variety of useful maximum heights (above the backing) of up to 3 mm, 1.5 mm, 1 mm, or 0.5 mm and, in some embodiments a minimum height of at least 0.05 mm, 0.1 mm, or 0.2 mm. The upstanding posts have a variety of aspect ratios (that is, a ratio of height to width at the widest point) such as at least about 2:1, 3:1, or 4:1. Advantageously, a variety of densities of the upstanding fastening elements may be useful. For example, the male fastening elements have a density of at least 248 per square centimeter (cm2) (1600 per square inch, in2) and up to about 1500/cm2 (10000/in2), 1240/cm2 (8000/in2), or 852/cm2 (5500/in2). For example, the density of the male fastening elements may be in a range from 271/cm2 (1750/in2) to about 852/cm2 (5500/in2) or from 248/cm2 (1600/in2) to 542/cm2 (3500/in2). The spacing of the male fastening elements need not be uniform.

Fastening components suitable for embodiments described herein may include laminates. In such an embodiment, the a fastening patch may be joined to a carrier, for example, by lamination (e.g., extrusion lamination), adhesives (e.g., pressure sensitive adhesives, hot melt adhesives, or structural adhesives), or other bonding methods (e.g., ultrasonic bonding, thermal bonding, compression bonding, or surface bonding).

In some embodiments the fastening component is joined to a carrier using surface bonding or loft-retaining bonding techniques. The term “surface-bonded” when referring to the bonding of fibrous materials means that parts of fiber surfaces of at least portions of fibers are melt-bonded to the backing of the fastening patch, on a side opposite the male fastening elements, in such a manner as to substantially preserve the original (pre-bonded) shape of the surface of the backing and to substantially preserve at least some portions of the surface of the backing in an exposed condition in the surface-bonded area. Quantitatively, surface-bonded fibers may be distinguished from embedded fibers in that at least about 65 percent of the surface area of the surface-bonded fiber is visible above the surface of the backing in the bonded portion of the fiber. Inspection from more than one angle may be necessary to visualize the entirety of the surface area of the fiber. The term “loft-retaining bond” when referring to the bonding of fibrous materials means a bonded fibrous material comprises a loft that is at least 80 percent of the loft exhibited by the material prior to, or in the absence of, the bonding process. The loft of a fibrous material as used herein is the ratio of the total volume occupied by the web (including fibers as well as interstitial spaces of the material that are not occupied by fibers) to the volume occupied by the material of the fibers alone. If only a portion of a fibrous web has the surface of the backing bonded thereto, the retained loft can be easily ascertained by comparing the loft of the fibrous web in the bonded area to that of the web in an unbonded area. It may be convenient in some circumstances to compare the loft of the bonded web to that of a sample of the same web before being bonded. In some of these embodiments, joining the fastening patch to a fibrous carrier comprises impinging heated gaseous fluid (e.g., ambient air, dehumidified air, nitrogen, an inert gas, or other gas mixture) onto a first surface of the fibrous web carrier while it is moving; impinging heated fluid onto the second surface of the backing while the continuous web is moving, wherein the second surface is opposite the male fastening elements; and contacting the first surface of the fibrous web with the second surface of the backing so that the first surface of the fibrous web is melt-bonded (e.g., surface-bonded or bonded with a loft-retaining bond) to the second surface of the backing. Impinging heated gaseous fluid onto the first surface of the fibrous web and impinging heated gaseous fluid on the second surface of the backing may be carried out sequentially or simultaneously. Further methods and apparatus for joining a continuous web to a fibrous carrier web using heated gaseous fluid may be found in U.S. Pat. Appl. Pub. Nos. 2011/0151171 (Biegler et al.) and 2011/0147475 (Biegler et al.).

Proximal and distal fastening components may include openings as illustrated for example in FIG. 4 of US Patent Application Publication No. US2014/0142533. Such may be in the form of a repeating pattern of geometric shapes such as polygons. The polygons may be, for example, hexagons or quadrilaterals such as parallelograms or diamonds. The openings may be formed in the fastening patch by any suitable method, including die punching. In some embodiments, the openings may be formed by slitting the thermoplastic backing of a fastening patch to form multiple strands attached to each other at intact bridging regions in the backing and separating at least some of the multiple strands between at least some of the bridging regions. The bridging regions are regions where the backing is not cut through, and at least a portion of the bridging regions can be considered collinear with the slits. The intact bridging regions of the backing serve to divide the slits into a series of spaced-apart slit portions aligned in the direction of slitting (e.g., the machine direction), which can be referred to as interrupted slits. In some embodiments, for at least some adjacent interrupted slits, the spaced-apart slit portions are staggered in a direction transverse to the slitting direction (e.g., the cross-machine direction). The interrupted slits may be cut into the backing between some pairs of adjacent rows of male fastening elements although this is not a requirement. In some embodiments, curved lines may be used, which can result in crescent shaped openings after spreading. There may be more than one repeating pattern of geometric shaped openings. The openings may be evenly spaced or unevenly spaced as desired. For openings that are evenly spaced, the spacing between the openings may differ by up to 10, 5, 2.5, or 1 percent. Further details about providing openings in a mechanical fastener can be found in U.S. Appl. Pub. No. 2012/0204383 (Wood et al.). In some embodiments, the fastening patch can comprise multiple strands attached to each other at intact bridging regions in the backing without spreading the strands apart to create openings. The interrupted slits may be made in either the longitudinal direction of the absorbent article or in a transverse direction. Such slits may improve the flexibility of the fastening patch improve the peel performance. Further details about providing interrupted slits in a mechanical fastener can be found in U.S. Appl. Pub. No. 2011/0313389 (Wood et al.).

As mentioned earlier, the proximal or distal fastening component can in some embodiments comprise multiple discrete fastening subcomponents. In some embodiments, the laminate comprises a plurality of narrow fastening patch strips separated by a distance that is usually smaller than the length of each fastening patch (that is, in the direction of the longest dimension of the carrier). An example of a configuration of two discrete fastening patches that effectively make up a fastening component is described in Int. Pat. Appl. Pub. No. WO 2011/163020 (Hauschildt et al.).

Fastening components may be attached to tab chassis 50 using any suitable method. For example, adhesives (e.g., pressure sensitive adhesives, hot melt adhesives, or structural adhesives), non-adhesive bonding (e.g., ultrasonic bonding, thermal bonding, compression bonding, or surface bonding as described above), or a combination of any of these methods may be useful

A portion of backsheet side 24 is also shown in FIG. 3. It includes a portion of landing zone area 8, which interfaces with fastening components 33 and 35. Landing zone area may comprise a discreet area, as shown, or backsheet side 24 may itself be formed of a material that suitably with the fastening tab's fastening component (e.g., a suitable non-woven material). In one embodiment it comprises a loop-type material. Any suitable loop type material may be used, so long as it provides appropriate engagement with the selected hook-type fastening component. For example, the hook-engaging surface of the loop-type material could be knit fabric, a nonwoven-type material (for example spunbound, meltblown, carded fiber, etc.). The loop could be single layer or multiple layer (laminated, for example, with a thermoplastic film).

Turning now to FIG. 4, a plan view rendering of a fastening tab according to one embodiment of the present invention is shown (in extended form where dashed lines 6 and 7 indicate the presence of creases that were previously folds). Fastening tab 3 is coupled to waist region 32 of a diaper (not fully shown) using a coupling system known in the art, as described above. Fastening tab in one embodiment has overall width “E”, proximal fastening component has width A; expansion area has width B, distal fastening component has width C, and lifting tab has width D.

In one preferred embodiment, width of different components is as follows: A and C are 20 mm, B is 35 mm, and D is 5 mm. In another preferred embodiment, A and C are 13 mm, B is 35 mm, D is 5 mm. Generally, A and C will be the same width, typically within about 10 mm to 30 mm, though other widths are possible. Width B is preferred to be within the range of about 10 mm to about 50 mm or more, and possibly as low as 8 mm. Below about 8 mm for width B and it becomes more sensible to just make a single fold rather than introduce a Z-fold.

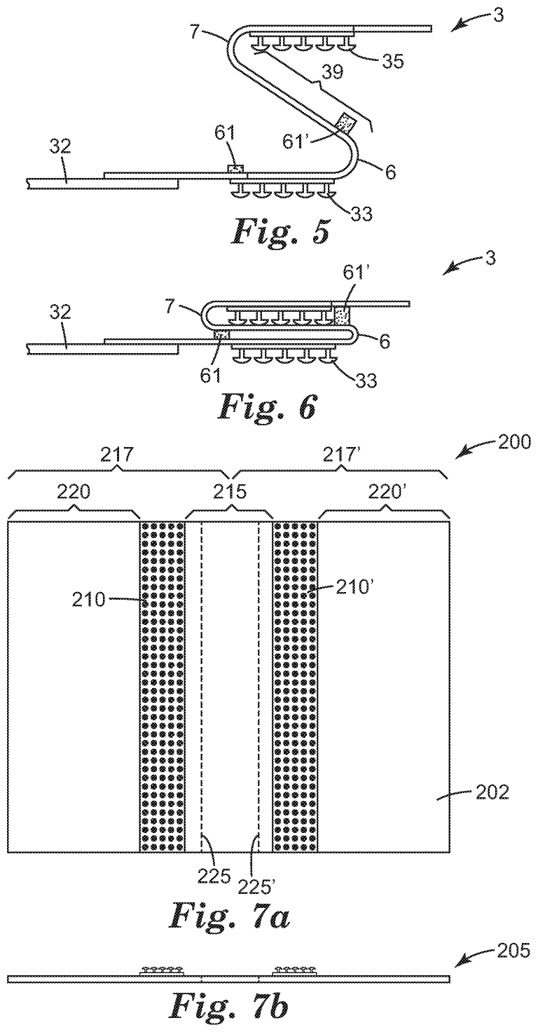

FIG. 5 is a profile view of the fastening tab 3 shown in FIG. 4, in a state between being folded and extended (FIGS. 4 and 6). In other words, it is partially unfolded. A proximal fold 6 and proximal fold 7 are shown in expansion area 39, the two folds combing to allow the tab chassis to double-back on itself. Optional adhesive area 61 and/or 61′ may be included to keep Z-folded fastening tab 3 in a folded configuration. In a fully folded state, the angles at fold 6 and 7 would approach about 180 degrees—that is, the material is folded backward onto itself. FIG. 6 is a profile view of the fastening tab shown in FIG. 4 and FIG. 5, in a Z-folded state.

Turning now to the manufacturing and conversion of the fastening tabs discussed hereto, a typical manufacturing process would be as follows. Two spaced apart lanes of hook are laminated to a carrier using ultrasonic welding or adhesive technology. The laminate is then folded in the machine direction using folding skis known in the art to create the z-fold geometry shown in figures included herein. This is then wound into a roll with the geometry shown in figure X2. When applying to diaper, the laminate in X2 is then s-cut to create right and left tabs. Figure X3 shows typical s-cut pattern. When the s-cut laminate is applied to the diaper, the right tab and the left tab will look slightly different since on the right tab, the finger lift will be on the top side of z-fold. For the left tab, the finger lift will be on the bottom side of the z-fold.

FIG. 7a is a combination plan view of an embodiment of a strip of fastening tab material 200 (before Z-folding along fold lines 225 and 225′. A first down-web strip of hook-type material 210 and a second down-web strip of hook-type material 210′ are coupled to carrier 202 and comprise the fastening components, oriented symmetrically relative to a central down-web axis (not shown in FIG. 7a). The hook-type material could be coupled to the carrier using known methods, for example by ultrasonic welding or by adhesive or integral formation. Expansion area 215 is the area between the two strips of hook-type material, which includes the places where folds that comprise the Z-fold will eventually be made. A first hemisphere 217 (left half) and a second hemisphere 217′ (right half) both include, in symmetry relative to each other, a fold line (225 or 225′), a strip of hook-type material (210 or 210′), and carrier area (220 or 220′).

FIG. 7b is a profile view of the fastening material 200 that is shown in FIG. 7a.

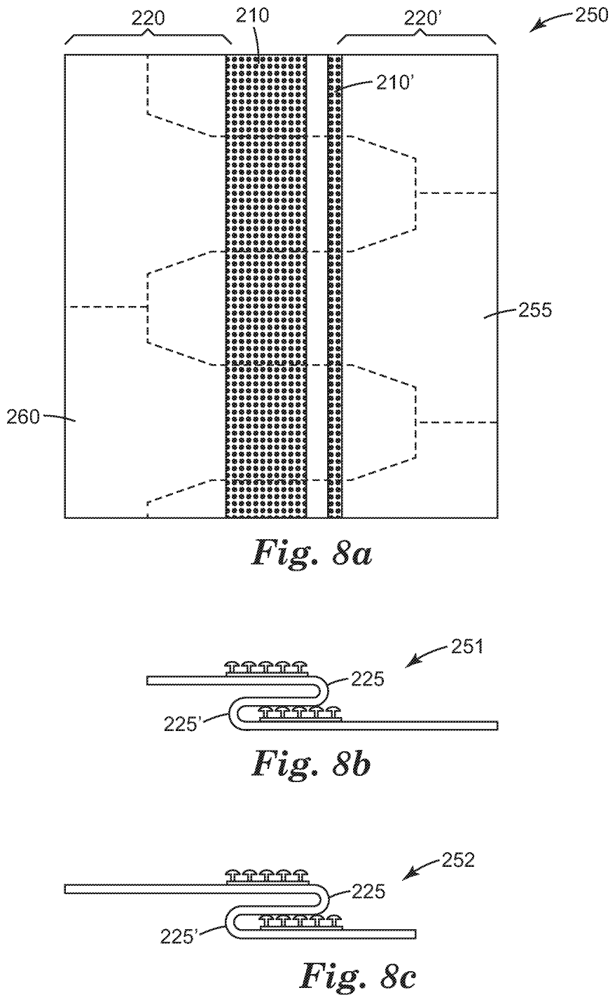

FIG. 8a shows the strip of fastening tab material of FIG. 7a after the introduction of a first and a second fold (together, a Z-fold) in expansion area 215 according to the fold lines 224 and 225′ shown in FIG. 7a. Z-folded fastening tab material 250 includes dotted lines defining a cutting pattern that will be used to S-cut individual fastening tabs 255 (which corresponds to profile view 251, in FIG. 8b) and fastening tab 260 (corresponding to profile view 252, in FIG. 8c). As can be seen, this pattern introduces a handedness aspect to the fastening tab, whereby carrier area 220 versus 220′ alternatively comprises the area to be bonded to the diaper chassis (essentially, the tabs are cut in a pattern for either the left or right side of the diaper assembly).

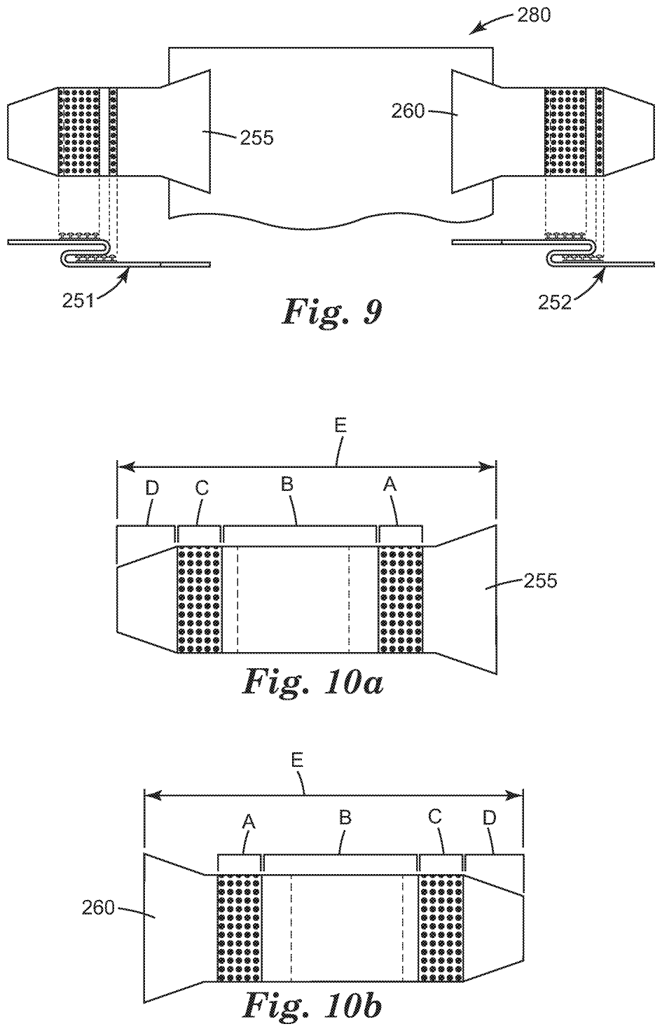

FIG. 9 shows the fastening tabs of FIGS. 7a and 8a in a diaper assembly 280, wherein the diaper tabs 255 and 260 have been coupled to diaper chassis 285 using techniques known in the art. Profile views 251 and 252 of the two fastening tabs are also shown, for reference.

FIG. 10 shows resulting fastening tabs after cutting per the plan shown in FIG. 10 (in expanded view—that is, no longer Z folded). Fastening tab 255 and 260 have an overall width “E”, a width for the proximal fastening component “A”, a width for expansion area “B”, a width for distal fastening component “B”, and a width for lifting tab “D” (which comprises the carrier area that extends beyond the distal fastening component). Widths for these dimensions is in accordance with the disclosure associated with FIG. 4. After folding, long strips of Z-folded manufacturing tab material would typically be wound onto a roll and shipped to diaper manufacturing facilities for conversion and assembly onto a diaper.

### Example

Two strips of hook material (20 mm wide) were laminated to a carrier material using a pressure sensitive adhesive, to create an embodiment resembling that shown in FIG. 7. The hook material was obtained from 3M Company, St. Paul, Minn., sold under the trade designation CS600 (of the general type described in U.S. Pat. No. 6,000,106). The carrier material was obtained from 3M Company, St. Paul, Minn., sold under the trade designation CLP06222 White Overlapping Fastener. The carrier material was 150 mm wide. The two hook strips, corresponding to elements 210 and 210′ in FIG. 7a, were laminated 37.5 mm from the outer edges of the carrier material to form the basic construction shown in FIG. 7a with a gap 215 of 35 mm between the two hook strips to form a laminate. The laminate was then folded twice between the hooks as shown by fold lines 225 and 225′ in FIG. 7a to create the Z-fold laminate shown in FIG. 8. Fold line 225 was located 4 mm from the inner edge of hook 210. Fold line 225′ was located 28 mm from the inner edge of hook 210. The Z-fold laminate was then S-cut as shown the dotted line in FIG. 8 to create left and right diaper tabs (diaper tabs 251 and 252). It should be noted that the S-cut line is running straight in the Cross-Direction through the folded area to avoid sharp edges at the fold lines.

1. An absorbent article having an inner surface, an outer surface, a first waist region, a second waist region, a crotch region extending longitudinally between and connecting the first waist region and the second waist region, the absorbent article comprising:
a chassis having longitudinally opposite ends, transversely opposite sides, a bodyside liner at least in part defining the inner surface of the article, an outer cover at least in part defining the outer surface of the article, and an absorbent core disposed between the liner and the outer cover;
a pair of Z-folded fastening tabs coupled to the transversely opposite ends of the chassis at the second waist region thereof, wherein the Z-folded fastening tabs, when unfolded, proximal and distal fastening components transversely separated from one another by an extension area, the fastening components coupled to a carrier.

2. The absorbent article of claim 1, wherein the Z-folded fastening tabs, when unfolded, comprise proximal and distal creases in the extension area, corresponding to folds that comprise the Z-fold.

3. The absorbent article of claim 1, wherein the carrier comprises a nonwoven material.

4. The absorbent article of claim 1, wherein the Z-folded fastening tabs comprise at least a first and a second fold in the extension area of each.

5. The absorbent article of claim 2, wherein the second folds are configured such that the fastening tab double-back on itself.

6. The absorbent article of claim 5, wherein the extension area separates the proximal and distal fastening components by at least 10 mm.

7. The absorbent article of claim 6, wherein the extension area separates the proximal and distal fastening components by at least 15 mm.

8. The absorbent article of claim 7, wherein the extension area separates the proximal and distal fastening components by at least 20 mm.

9. The absorbent article of claim 8, wherein the extension area separates the proximal and distal fastening components by at least 25 mm.

10. The absorbent article of claim 9, wherein the extension area separates the proximal and distal fastening components by at least 30 mm.

11. The absorbent article of claim 6, wherein the fastening tab extends transversely beyond the distal fastening components to define a lifting tab comprising carrier material.

12. The absorbent article of claim 1, wherein the proximal and distal fastening components comprise an area of hook-type fastener material.

13. The absorbent article of claim 1, wherein the proximal fastening component comprises a plurality of transversely separated strips of hook-type material.

14. The absorbent article of claim 1, wherein the distal fastening component comprises a plurality of transversely separated strips of hook-type material.

15. The absorbent article of claim 1, wherein the outer cover of the chassis comprises at least one landing zone comprising loop-type material, positioned at the first waist region to fastenably engage with the proximal and distal fastening components of the pair of fastening tabs.

16. The absorbent article of claim 1, wherein the fastening tabs further comprise areas of pressure sensitive adhesive which hold the Z-folded fastening tabs in a Z-folded state.

17. An fastening tab of an absorbent article, the fastening tab having longitudinal opposite ends and transversely opposite ends, comprising a non-woven backing material having proximal and distal fastening components bonded thereto and transversely spaced apart by a separation zone at least 10 mm wide, and wherein the fastening tab is folded at least two times in the separation zone.

18. The fastening tab of claim 17, wherein the fastening tab is folded in a Z-shape.

19. The fastening tab of claim 17, wherein the fastening tab includes areas of pressure sensitive adhesive that releasably hold the folded Z-shape.

20. A strip of non-woven material which, when S-cut, may be used for the fastening tab region of an absorbent diaper or adult incontinence device, the nonwoven material comprising:
a strip of non-woven backing material having a longitudinal and transverse dimension, the left and right hemispheres longitudinally;
left hemisphere fastening component coupled to the left hemisphere of the strip of non-woven backing material;
right hemisphere fastening components coupled to the right hemisphere of the strip of non-woven backing material;
wherein the left and right hemisphere fastening components area separated by an expansion area and are symmetric to one another.

21. The strip of claim 20, wherein the left hemisphere expansion area comprises a first and a second fold running longitudinally along the length of the strip.

22. The strip of claim 21, wherein the first and second folds in the left hemisphere comprise a Z-fold.

23. The strip of claim 22, wherein the right hemisphere expansion area comprises a first and a second fold running longitudinally along the length of the strip.

24. The strip of claim 20, wherein the right hemisphere expansion area comprises a first and a second crease.

25. The strip of claim 22, wherein the first and second folds in the right hemisphere comprise a Z-fold.

26. The strip of claim 20, wherein the fastening component comprise a hook-type material.

27. The strip of claim 20, further comprising a strip of pressure sensitive adhesive positioned adjacent the strip of either fastening component.
